(19) United States
(12) Patent Application Publication
(10) Pub. No.: US 2004/0058310 A1
(43) Pub. Date: Mar. 25, 2004

(54) Method for measuring a marker indicative of the exposure of a patient to nicotine; a kit for measuring such a marker

(76) Inventors: Regls Christian Grailhe, Paris (FR); Anne Cormier, Paris (FR); Jean Pierre Changeux, Paris (FR); Gilbert Lagrue, Paris (FR)

Correspondence Address: FINNEGAN, HENDERSON, FARABOW,, GARRETT & DUNNER, L.L.P., 1300 I Street, NW, Washington, DC, 20005-3315, US

(21) Appl. No.: 10/247,585

(22) Filed: Sep. 20, 2002

(51) Int. Cl. A61K 51/00; C12Q 1/00

(52) U.S. Cl. 435/4; 424/1.11

(57) ABSTRACT

The present invention demonstrates that epibatidine binds to nicotinic receptors on leukocytes. Epibatidine is a sensitive detector of increases in the number of nicotinic receptors expressed by a cell. Epibatidine binds to the leukocytes of smokers, but does not bind to the leukocytes of non-smokers. Both in vivo and ex vivo nicotine exposure induces epibatidine receptor expression. Epibatidine binding to leukocytes reflects epibatidine binding in the central nervous system, which in turn reflects nicotine-induced effects on the central nervous system. Epibatidine binding assays in peripheral blood leukocytes can be used to evaluate an individual's exposure to, and dependence on, nicotine.

### BACKGROUND OF THE INVENTION

1. Field of the Invention

The present invention relates to epibatidine binding to leukocytes in smokers. The present invention also relates to a method of detecting epibatidine binding to leukocytes in smokers.

2. Description of Related Art

Tobacco Use and Addiction

The morbidity and mortality associated with tobacco use underlies the need to understand tobacco addiction and to develop effective therapeutics for decreasing dependence on tobacco use, including smoking cessation therapies.

Tobacco kills more people than alcohol, traffic accidents, and AIDS combined, as a result of cancers, heart disease and respiratory disease (Slama 1998). Although most smokers understand the health risks of tobacco use, they continue to smoke. Physiological addiction is associated with tobacco use in many smokers. The nicotine present in tobacco smoke is mainly responsible for tobacco addiction, which drives many people to consume tobacco (Dani, Ji et al. 2001) (Benowitz 1992). The rewarding effects of nicotine occur in the central nervous system (CNS), by modulating neuronal excitability and synaptic communications (Albuquerque, Alkondon et al. 1997; Wonnacott 1997; Dani, Ji et al. 2001).

The Fagerström Tolerance Test for Nicotine Dependency allows health care professionals to classify smokers according to their level of nicotine dependency and to identify those most likely to need nicotine replacement therapy in order to successfully stop smoking (Fagerström et al. 1991). The test is comprised of a series of questions directed to the smoker's tobacco use. It includes the questions “How many cigarettes per day do you smoke?” and “How soon after you wake up do you smoke your first cigarette?” The answers are analysed to generate a composite score; a high score indicates that physiological addiction is likely to be present, and the smoker will require nicotine replacement therapy.

Nicotinic Acetylcholine Receptors

Acetylcholine is a neurotransmitter that is released from cholinergic nerve axons in response to a calcium-mediated stimulus. Acetylcholine mediates different responses depending upon the type of cholinergic receptor it encounters. Acetylcholine receptors are classified as either nicotinic or muscarinic. The response of most autonomic effector cells in peripheral visceral organs is typically muscarinic, whereas the response in parasympathetic and sympathetic ganglia, as well as the responses of skeletal muscle, is nicotinic.

Neuronal nicotinic acetylcholine receptors (nAChRs) are members of the excitatory ligand-gated cation channel family. They are derived from 12 gene products termed α2-α10 and β2-β4 (Role and Berg 1996; Albuquerque, Alkondon et al. 1997; Lindstrom 1997), which provide raw materials for the assembly of several receptor isoforms. Molecular biological studies have demonstrated heterogeneity in the composition of neuronal nicotinic receptors in both brain and periphery. Diverse ranges of compounds are known to be pharmacologically active at nAChRs.

In humans, neuronal nAChRs can be divided by their radioligand binding properties into two classes. The first is a class with [¹²⁵I]-α-bungarotoxin ([¹²⁵I]-α-bgt) binding sites specific to homomeric α7 nAChRs. The second is a class with[³H]-nicotine and [³H]-epibatidine binding sites specific to heteromeric nAChRs. Immunoprecipitation studies indicate that >90% of high affinity nicotinic agonist binding in the rat brain corresponds to receptors composed of α4 and β2 subunits (Flores, Rogers et, al. 1992). In the central nervous system, the most abundant forms of nicotinic receptors are the α₄β₂ receptor and the α-bungarotoxin-sensitive homopentameric receptor α7 (Léna and Changeux 1997; Lukas, Changeux et al. 1999).

In humans, a role for nicotinic receptors in smoking addiction can be implied, but is not yet well understood. Such knowledge might contribute to the understanding of the complexity of smoking dependence and to enable effective treatments.

### Radiolabeled Nicotine and Epibatidine and Receptor Binding

Epibatidine was first isolated by Daly et al. from the skin of the Ecuadoran poison frog, Epipedobates tricolor (Daly et al 1980). Its structure was determined by mass spectroscopy, infrared spectroscopy, and nuclear magnetic resonance as exo-2(6-chloro-3-pyridyl)-7-azabicyclo[2.2.1]-heptane (Spande et al. 1992). This alkaloid has been shown to be a potent analgesic with a nonopioid mechanism of action. The analgesic effect of epibatidine was approximately 200 times higher than morphine using the hot plate assay, and approximately 500-fold that of morphine in eliciting the Straub-tail response. The epibatidine-induced analgesia was not blocked by the opioid receptor antagonist naloxone. Furthermore, it has been determined that epibatidine had a negligible affinity for the opioid receptor (1/8000 times that of morphine). See, Spande, et al., J. Am. Chem. Soc., 114:3475 (1992). Thus, epibatidine is a highly potent and effective analgesic, with far less potential for addiction and tolerance than morphine.

Epibatidine binds to, and activates, nicotinic acetylcholine receptors. It is effective at very low concentrations; the Kᵢ=0.043-0.055 nM, i.e. about 55 pM. This binding can be blocked by mecamylamine, a noncompetitive nicotinic antagonist.

Synthetic analogues of epibatidine have been synthesized. One of these, epiboxidine, has analgesic and cognitive-enhancing properties (Qian 1993). Epiboxidine is less potent than epibatidine, but also less toxic. The affinity of epiboxidine (Kᵢ=0.6 nM) for the nAChR is higher than that of nicotine (Kᵢ=1.01 nM). Other synthetic analogues of epibatidine include homoepibatidine, bis-homoepibatidine, and an azabicyclooctane analogue (Xu et al. 1996a; Xu et al. 1996b; Malpass et al. 1996; Zhang et al. 1997).

Studies performed on brain slices from smokers and matched controls reveal that smokers' brains bind more [³H]-nicotine and [³H]-epibatidine binding sites than non-smokers (Benwell, Balfour et al. 1988; Breese, Marks et al. 1997; Perry, Davila-Garcia et al. 1999; Paterson and Nordberg 2000). Nicotine binding sites were increased in the hippocampus and thalamus of smokers by a factor of 1.5 to 3 (Perry, Davila-Garcia et al. 1999). In rodents, chronic in vivo nicotine treatment was reported to up-regulate the numbers of brain nAChRs binding sites in a concentration-dependant manner (Marks, Stitzel et al. 1985; Schwartz, K. J. et al. 1985; Flores, Rogers et al. 1992). After cessation of nicotine treatment, nAChR levels returned to control values (Schwartz, K. J. et al. 1985) (Marks, Stitzel et al. 1985), demonstrating the reversibility of this phenomenon.

In humans, far less is known about the relationship between nicotine abuse and receptor levels. However, the number of [³H]-nicotine binding sites is positively correlated with the number of cigarettes smoked per day (Benwell, Balfour et al. 1988) (Breese, Marks et al. 1997). In the human brain, modifications in neuronal nAChRs are very difficult to investigate in vivo during smoking and/or smoking cessation.

Recently, nAChRs have been identified on human blood lymphocytes (Sato, Fujii et al. 1999) and polymorphonuclear leukocytes (Benhammou, Lee et al. 2000). Correlations were established between the number of [³H]-nicotine binding sites and the number of cigarettes smoked per day. Some studies have revealed the presence of some functional neuronal nAChRs in non-neuronal cells, such as epithelial cells (Maus, Pereira et al. 1998), keratinocytes (Grando, Zelickson et al. 1995), and endothelial cells (Macklin, Maus et al. 1998).

Notwithtanding the progress in the art, there exists a need for increased understanding of the biological processes involved in tobacco use and tobacco addiction. Increased knowledge of these processes would be useful in the diagnosis, treatment, and prevention of tobacco use and addiction.

### SUMMARY OF THE INVENTION

The present invention addresses the need in the art for a better understanding of human nicotinic receptors involved in tobacco use and tobacco addiction. Nicotine induces up-regulation of polymorphonuclear leukocyte nicotinic receptors. Thus, [³H]-epibatidine binding to leukocytes provides a method for tracking plastic changes in nicotinic receptors, and reflects changes that occur in the central nervous system as well as in the periphery. This method is useful for determining whether a patient is a smoker or a non-smoker, the degree of a patient's tobacco use, and the necessity for nicotine replacement therapy as part of the patient's smoking cessation program. The profile of [³H]-epibatidine binding sites on blood cells reflects physical dependence on nicotine and supplies help to the health care professional in determining an optimal smoking cessation treatment.

The present invention demonstrates for the first time that epibatidine binds to nicotinic receptors of leukocytes.

The present invention also shows for the first time that epibatidine detects an increase in the nicotinic receptor binding sites in smokers compared to non-smokers. Epibatidine is a sensitive detector of increases in nicotinic binding sites. Epibatidine binding is not observed in non-smokers. The level of epibatidine binding and the amount of tobacco use is tightly correlated.

Epibatidine binding sites are present in smoker's leukocytes, but not in the leukocytes of non-smokers. Therefore, epibatidine binding to leukocytes present in a sample of the subject's blood can be used to establish whether the subject is smoker or a non-smoker.

The degree of epibatidine binding to smokers' leukocytes is correlated with the degree of tobacco use. Therefore, epibatidine binding to leukocytes present in a sample of the subject's blood can also be used to evaluate a subject's tobacco use.

The degree of epibatidine binding to smokers' leukocytes is correlated with the degree of tobacco addiction, as measured by the Fagerström test. Therefore, in addition, epibatidine binding to leukocytes present in a sample of the subject's blood can be used to study tobacco addiction.

The degree of epibatidine binding to smokers' leukocytes is correlated with the degree of nicotine exposure. Therefore, epibatidine binding to leukocytes present in a sample of the subject's blood can be further used to study the effects of passive smoke exposure.

The degree of epibatidine binding to smokers' leukocytes is correlated with the degree of tobacco use. Therefore, epibatidine binding to leukocytes present in a sample of the subject's blood can be used to monitor diseases or conditions associated with tobacco use.

Since epibatidine binding profiles in leukocytes reflects a physical dependence on tobacco, epibatidine binding to leukocytes present in a sample of the subject's blood can also be used to design-effective smoking cessation programs.

### BRIEF DESCRIPTION OF THE DRAWINGS

This invention will be described with reference to the drawings in which:

FIG. 1: Saturation of specific [³H]-epibatidine (A) and [¹²⁵I]-α-bungarotoxin (B) binding to human polymorphonuclear cells. Insets, Scatchard analyses of specific binding of [³H]-epibatidine and [¹²⁵I]-α-bungarotoxin (1 pM to 10 nM). Nonspecific binding was determined in the presence of 1 μM or 100 μM of nicotine respectively. Data are the mean± standard error of three experiments.

FIG. 2: [³H]-epibatidine (A) and [¹²⁵I]-α-bungarotoxin (B) binding sites in human polymorphonuclear cells isolated from smokers (n=90 and 60, respectively) and non-smokers (n=50). Binding levels were measured at 10 nM of [³H]-epibatidine and [¹²⁵I]-α-bungarotoxin. Nonspecific binding was determined in the presence of 1 μM or 100 μM of nicotine, respectively. Data are the mean± standard error of three experiments. ** p<0.001.

FIG. 3: Ex vivo up-regulation of [³H]-epibatidine binding sites on isolated human polymorphonuclear cells. Human cells were incubated for three days at 4° C. in the presence or absence of 1 mM nicotine. Binding levels were measured at 10 nM of [³H]-epibatidine and [¹²⁵I]-α-bungarotoxin. Nonspecific binding was determined in the presence of 1 μM or 100 μM of nicotine, respectively. Data are the mean± standard error of three experiments. * p<0.05, ** p<0.001.

FIG. 4: Correlation analysis of smoking history with [³H]-epibatidine binding sites. Correlations were observed with the Fagerström test. (A) represents the number of cigarettes smoked per day and (B) represents the delay between morning awakening and the first cigarette smoked (C).

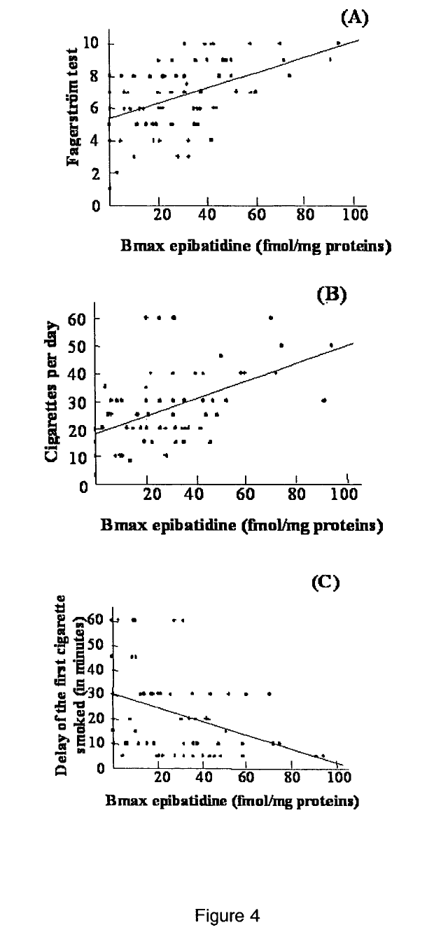

### DETAILED DESCRIPTION OF THE INVENTION

This invention identifies and quantifies differences in peripheral or neuronal nicotinic receptors between populations of smokers and non-smokers. This invention quantifies receptor binding sites using epibatidine. In a preferred embodiment, [³H]-epibatidine and [¹²⁵I]-α-bungarotoxin radioligands are used.

As used herein, leukocytes are white blood cells. Five types of leukocytes are normally present in the blood. These are traditionally divided into two groups, based on their nuclear shape and cytoplasmic granules. Granulocytes have multilobed nuclei and prominent granules in their cytoplasm. Mononuclear leukocytes have non-lobulated nuclei and less prominent cytoplasmic granules. Granulocytes include neutrophils, eosinophils, and basophils. Mononuclear leukocytes include lymphocytes and monocytes.

The multilobed nucleus of granulocytes can assume many morphological shapes, leading to the term polymorphonuclear leukocytes (PMN). As used herein, polymorphonuclear leukocytes are granulocytes, and include neutrophils, basophils, and eosinophils.

Nicotine induces up-regulation of PMN nicotinic receptors. Thus, [³H]-epibatidine binding to PMN reflects nicotinic receptor plastic changes that occur in the central nervous system and are related to tobacco consumption. Therefore, the nicotinic receptor profile present on PMN reflects a person's physiological state with respect to tobacco use. Nicotinic receptor labelling provides information about whether a patient is a smoker or non-smoker, and provides information about the degree of tobacco use. In addition, it provides a mechanism to study the effects of passive exposure to tobacco smoke (“second-hand smoke”). Also, it provides a method to monitor the risk of a patient contracting a tobacco-related disease or condition.

This information can be used to design optimal smoking cessation therapies, since [³H]-epibatidine binding profiles in PMN reflects a physical dependence to tobacco.

The presence of nAChRs in B lymphocytes was further confirmed by studying the [³H]-epibatidine and [¹²⁵I]-α-bungarotoxin binding. Epibatidine is a potent agonist of heteromeric nAChRs, while -α-bungarotoxin (α-Bgt) binds to both muscle-type nAChRs and homomeric neuronal-type of nAChRs. In addition, epibatidine penetrates inside the cell and binds both surface and intracellular receptors, while α-Bgt, when tested at ice-cold temperature, binds only surface-expressed receptors. Normal B lymphocytes and melanoma X63-Ag8 cells contained almost equal amounts of total epibatidine-binding sites per cell (12,220±3,200 and 10,170±1,100, respectively, means and standard error of three independent experiments).

By “patient” it is meant any living animal, including, but not limited to, a human who has, or is suspected of having or being susceptible to, a disease or disorder, or who otherwise would be a subject of investigation relevant to nicotine use. Accordingly, a patient can be an animal that has been bred or engineered as a model for nicotine use, tobacco use, tobacco addiction, or any other disease or disorder. Likewise it can be a human suffering from, or at risk of developing, a disease or disorder associated with tobacco use, or any other disease or disorder. Similarly, a patient can be an animal (such as an experimental animal, a pet animal, a farm animal, a dairy animal, a ranch animal, or an animal cultivated for food or other commercial use) including a human, who is serving as a healthy control for investigations into diseases and/or disorders associated with tobacco use, or any other disease or disorder.

As used herein, the term “marker indicative of the exposure of a patient to nicotine” refers to a marker which level depends on the dose of nicotine to which a patient is exposed. In a preferred embodiment, a marker is a leukocyte neuronal nicotinic receptor site which binds to epibatidine.

Exposure to nicotine can come from smoking cigarettes, chewing tobacco, nicotine patches, beverages, gums, or passive smoking, for example.

As used herein, epibatidine refers to a molecule such as the one isolated by Daly et al., but may also refer to any natural or synthetic analogue of epibatidine, any molecule derived from epibatidine, or any other molecule which is capable of binding to the epibatidine binding sites of neuronal nicotinic receptors and shows correlation with the Fagerström Test.

Various means known to one skilled in the art can be used for measuring the binding of epibatidine to leukocytes. For example, epibatidine can be labeled; it can be radiolabelled or labeled with a fluorescent element. Any other method known by one skilled in the art, such as ELISA, can also be used.

The state of neuronal nicotinic receptors is reflected in the state of nicotinic receptors in leukocytes, for example in PMN. Studies of smoker and nonsmoker populations, using [³H]-epibatidine and [¹²⁵I]-α-bungarotoxin ligands reveals differences in the nicotinic receptor profile between the groups. Binding studies can determine a profile of leukocyte binding sites for these ligands, which includes a quantitative count of the number of binding sites, as well as a quantitative estimate of the affinity of each ligand to each class of binding site. The ligands can be labelled to facilitate the detection and/or measurement of ligand/receptor binding. Preferably the ligands are radiolabeled. Generally, ligand binding assays are performed by placing ligand, for example, radiolabeled ligand, in proximity to isolated leukocytes, for example PMN, permitting the ligand to bind to its specific receptor, then separating the leukocytes with bound ligand from free, unbound ligand.

This invention shows that epibatidine binding sites were present in smoker's leukocytes, such as PMN, but not in the leukocytes of non-smokers. α-Bungarotoxin binding sites were found in both smokers and non-smokers leukocytes, such as PMN, and thus can serve as an experimental control. The induction of additional nicotinic receptor binding sites in leukocytes, such as PMN, following tobacco use reflects a long-term adaptation of the brain nicotinic receptor that has been chronically exposed to nicotine. Such information is easily accessible to health care professionals.

The invention provides for a method of measuring the amount of epibatidine, a marker that indicates a patient's exposure to nicotine, present in a patient's leukocytes. Epibatidine binds to neuronal nicotinic receptors in the central nervous system that are up-regulated by tobacco use, and are involved in mediating nicotine addiction. The level of epibatidine binding to nicotinic receptors on leukocytes provides a readily accessible clinical method for monitoring epibatidine binding to the clinically inaccessible neuronal nicotinic receptors in the central nervous system.

The level of epibatidine bound to a patient's leukocytes establishes whether the patient is a smoker or a non-smoker, and reflects the quantitative level of the patient's tobacco use. The level of epibatidine bound to a patient's leukocytes also establishes the degree of tobacco dependence. The level of epibatidine bound to a patient's leukocytes further establishes the degree of a patient's passive smoke exposure. Epibatidine binding to leukocytes present in a blood sample is useful for monitoring diseases or conditions associated with tobacco use. Epibatidine binding to leukocytes present in a blood sample can also form the basis for the design of a smoking cessation program.

A kit for measuring the level of epibatidine binding to leukocytes can be used to establish whether a patient is a smoker or a non-smoker, the degree of tobacco use, the level of addiction, the degree of exposure to passive smoke, and to monitor diseases or conditions associated with tobacco use. The kit can be used as the basis for a smoking cessation program.

A detectable tag or marker can be attached to epibatidine, or a synthetic analogue of epibatidine, to render the molecule detectable by conventional methods of detection. Epibatidine or a synthetic analogue can be radiolabelled with a radioisotope, for example tritium (³H).

This invention will be described in greater detail in the following Examples.

### EXAMPLE 1

### Isolation of Polymorphonuclear Leukocytes from Human Blood

Human polymorphonuclear leukocytes were isolated according to a slightly modified version of the method described by Cabanis (Cabanis, Gressier et al. 1994). Briefly, 20 ml of fresh heparinized blood were diluted with an equal amount of phosphate-buffered saline (PBS) 0.1 M, pH 7.4, and placed above 10 ml of Histopaque-1077. After centrifugation at 400 g for 30 min, the pellet was resuspended in 40 ml of cold isotonic ammonium chloride solution (NH₄Cl 0.15 M, NaHCO₃ 10 mM). Following 20 min at 4° C., the cell suspension was centrifuged at 160 g for 10 minutes, and the white pellets were washed twice in 10 ml of Hank's buffer. The protein content was measured using the method developed by Lowry et al. (Lowry et al. 1951).

### EXAMPLE 2

### Radioligand Binding Assays

Binding assays were performed on intact purified PMN. Cellular protein (100 μg) was incubated with 10 nM [³H]-epibatidine for 30 min. or with [¹²⁵I]-α-bungarotoxin for 60 min at 25° C. in a volume of 100 μl. Specific binding was defined as the difference between total binding and binding in the presence of 1 μM or 100 μM nicotine, performed in triplicate.

Saturation studies were conducted with increasing concentrations of [³H]-epibatidine (1 pM-10 nM) and [¹²⁵I]-α-bungarotoxin (1 pM-10 nM). Specific binding was defined as the difference between total binding and binding in the presence of 1 μM or 100 μM nicotine, respectively, performed in triplicate. Following the binding reactions, bound and free ligands were separated by rapid vacuum filtration through Whatman GF/B fiberglass filters (Polylabo) treated with ice-cold buffer (KH₂PO₄ 5 mM, Na₂HPO₄ 20 mM, NaCl 100 mM, pH 7.4) containing 0.1% milk. The filters were rinsed three times with 5 ml of the same ice-cold buffer and placed in vials with 4 ml of Picofluor 30 scintillation liquid (Packard Instrument). The radioactivity was determined by liquid scintillation counting.

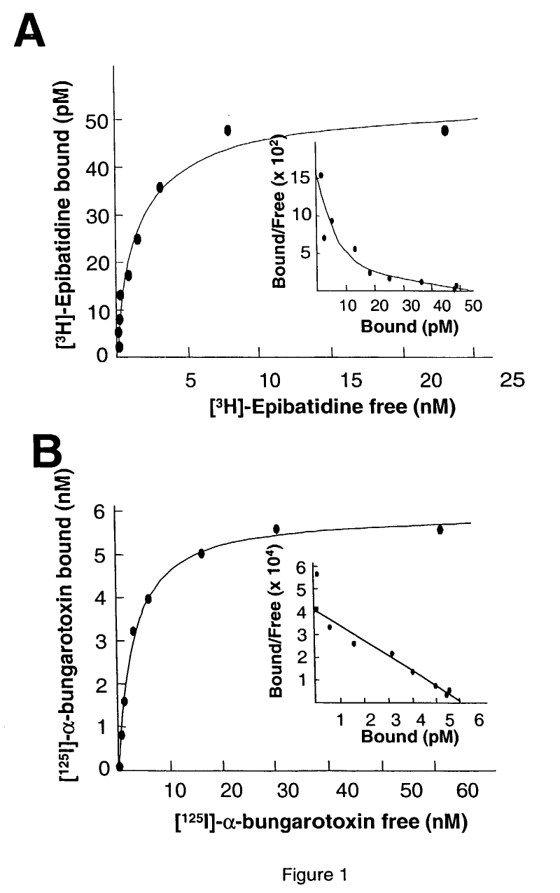

Binding experiments were investigated in smokers' polymorphonuclear leukocyte cells in presence of various concentrations of [³H]-epibatidine ranging from 0.1 nM to 25 nM. Saturation levels and Scatchard plots show the presence of [³H]-epibatidine binding sites (FIG. 1A). The Scatchard plots are biphasic with a Hill coefficient of 0.76, characteristic of the presence of two binding sites. The first site has a high affinity (Kd₂=2.11±0.43 nM) and represents 86.86% of the total binding sites (Bmax₁=46.97±5.64 fmol/mg protein). The second site has a very high affinity (Kd₁=56.3±27.8 pM) and represents 14.14% of the total binding sites (Bmax₁=7.73±0.64 fmol/mg protein).

Smokers' polymorphonuclear leukocytes (n=3) were used to investigate saturation experiments. The linearity of the Scatchard plot (nH=1.08±0.06) indicates a single class of nicotine binding sites with an apparent Kd=2.77±1.54 nM and Bmax=189.46±126.27 fmol/mg proteins (FIG. 1B).

[¹²⁵I]-α-bungarotoxin binding sites were present in the PMN of both smokers and non smokers. Approximately 30% of the PMN tested in each population demonstrated the presence of [¹²⁵I]-α-bungarotoxin binding sites. Unexpectedly, the binding studies described above revealed that [³H]-epibatidine was only present in smoker's PMN, and was not present in the PMN of non-smokers.

Notably, following an ex-vivo nicotine exposure lasting several days, nicotine induced the formation of [³H]-epibatidine binding sites in non smokers PMN. RT-PCR with PMN mRNA extract revealed the induced expression of α3, α4 and β2 subunits.

### EXAMPLE 3

### Comparison of Smokers With Non-Smokers

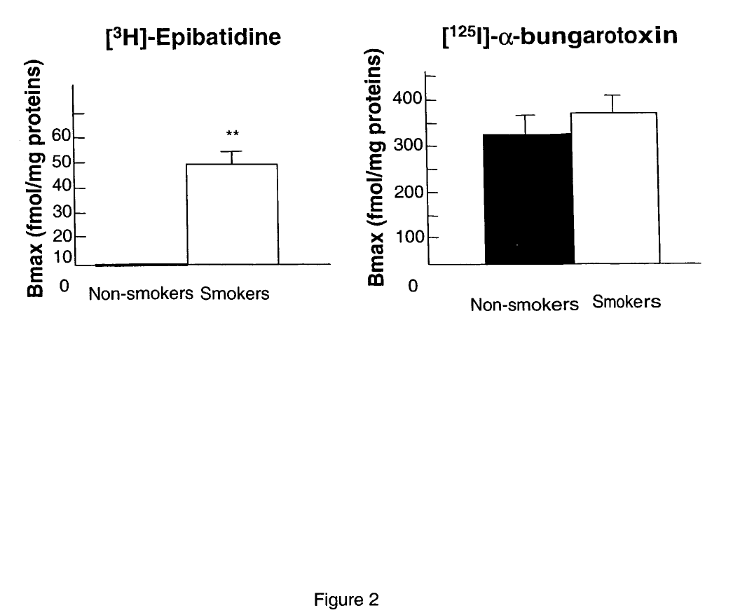

Nicotinic receptor binding sites in PMN were investigated from 90 smokers and 50 non-smokers. Participating smokers were recruited from the Centre de Tabacologie, Hôpital A. Chenevier, Créteil. As shown in FIG. 2, [³H]-epibatidine binding sites were detected in 82% of the smokers. In smokers, the total number of binding sites was 40.65±4.89 fmol/mg protein, and ranged from 2.87 to 94.94 fmol/protein. None of the 50 non-smokers tested had any detectable [³H]-epibatidine binding sites. No difference was observed between [¹²⁵I]-α-bungarotoxin binding sites in smokers and non-smokers.

### EXAMPLE 4

### Ex-Vivo Nicotine Up-Regulation

To compare the dependency for nicotine-induced upregulation of [³H]-epibatidine binding sites, binding assays were performed on purified PMN with or without chronic nicotine treatment. Freshly purified polymorphonuclear leukocyte blood cells were incubated with or without 1 mM nicotine for three days at 4° C. in a final volume of 1 ml. On the fourth day, cells were washed twice in 15 ml Hank's buffer to eliminate the presence of nicotine.

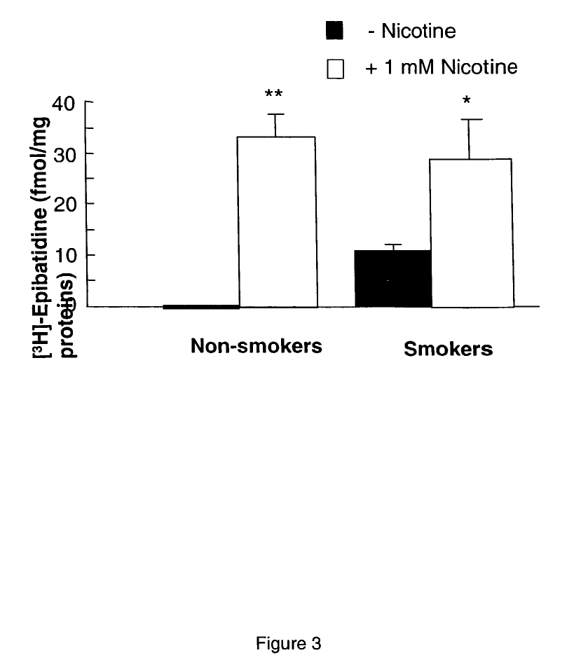

The exogenous presence of nicotine (1 mM) increased the number in [³H]-epibatidine binding sites on smokers' PMN 2.67-fold, from 10.92±02 to 29.19±10.28 fmol/mg protein (FIG. 3). Interestingly, in non-smokers' PMN, it was observed that the same number of [³H]-epibatidine binding sites were present as in the smokers' PMN (Bmax of 34.66±6.25 fmol/mg protein).

Nicotine induced no change in the binding of [¹²⁵I]-α-bungarotoxin to PMN.

### EXAMPLE 5

### RT-PCR and Southern Blot Analysis

Total RNA was isolated from smokers and non-smokers (TRIZOL, Gibco). First strand cDNA synthesis reactions were performed after annealing 5 μg of total RNA with 100 ng random hexamers (70° C. for 10 minutes) by incubation at 42° C. for 50 minutes. Polymerase chain reactions were carried out in a 20 μl reaction volume containing 1 μl cDNA product, 250 ng of both forward and reverse primers (Table 1), 5 units of Taq polymerase then cycled 30 times at 95° C. for 1 minute, 55° C. for 1 minute and 72° C. for 2 minutes. The amplified DNA products were analysed by agarose gel electrophoresis and stained with ethidium bromide. The PCR products were transferred from the gel to Hybond-N⁺ membranes (Amersham) and hybridised with a ³²P-end-labelled oligoprobe (Table 1). Southern blot hybridizations were performed overnight at 42° C. in hybridisation buffer (5×SSC, 1× Denhardt's, 20 mM sodium phosphate buffer pH 6.5, 0.1% SDS, 100 μg/ml tRNA). The Southern blots were washed for 1 hour at room temperature in 2×SSC and 1% SDS, then exposed to X-ray film (Kodak Biomax) overnight with an intensifying screen.

### EXAMPLE 6

### Statistical Analysis

For each experiment, the mean values of RCR or percentages were compared in a one-way analysis of variance and a Dunneft test. EC₅₀ was calculated by non-linear regression fit of effect-concentration (C) curve to the equation: E=(Eₘₐₓ×C)/(C+EC₅₀), where Eₘₐₓ and EC₅₀ are the maximal efficiency and the concentration producing 50% effect respectively, using commercially available software (Micropharm®) (Urien 1995). Data from binding experiments were analysed by means of non-linear regression with commercially available software (Micropharm®) (Urien 1995).

### REFERENCES

The specification is most thoroughly understood in light of the following references, all of which are hereby incorporated in their entireties.

Albuquerque, E., M. Alkondon, et al. (1997). “Properties of neuronal nicotinic acetylcholine receptors: pharmacological characterization and modulation of synaptic function.” Journal of Pharmacology & Experimental Therapeutics. 280(3): 1117-1136.

Benhammou, K., M. Lee, et al. (2000). “[(3)H]Nicotine binding in peripheral blood cells of smokers is correlated with the number of cigarettes smoked per day.” Neuropharmacology 39(13): 2818-29.

Benowitz, N. L. (1992). “Cigarette smoking and nicotine addiction.” Med Clin North Am 76(2): 415-37.

Benwell, M., D. Balfour, et al. (1988). “Evidence that tobacco smoking increases the density of (−)-[3H]nicotine binding sites in human brain.” Journal of Neurochemistry 50(4): 1243-7.

Breese, C. R., M. J. Marks, et al. (1997). “Effect of smoking history on [3H]Nicotine binding in human post-mortem brain.” The Journal of Experimental and Clinical Therapeutics 50: 1243-1247.

Cabanis, A., B. Gressier, et al. (1994). “A rapid density gradient technique for separating polymorphonuclear granulocytes.” APMIS 102: 119-121.

Daly et al., (1980) J. Am. Chem Soc., 102:830.

Dani, J. A., D. Ji, et al. (2001). “Synaptic plasticity and nicotine addiction.” Neuron 31(3): 349-352.

Fagerström, K. O., Heatherton, T. F., Kozlowski, L. T. (1991) “Nicotine addiction and its assessment.” Ear Nose Throat J. 69: 763-765.

Flores, C. M., S. W. Rogers, et al. (1992). “A subtype of nicotinic cholinergic receptor in rat brain is composed of alpha4-subunit and beta2-subunit and is up-regulated by chronic nicotine treatment.” Mol. Pharmacol. 41(1): 31-37.

Grando, S. A., B. D. Zelickson, et al. (1995). “Keratinocyte muscarinic acetylcholine receptors: immunolocalization and partial characterization.” Journal of Investigative Dermatology 104(1): 95-100.

Léna, C. and J. P. Changeux (1997). “Pathological mutations of nicotinic receptors and nicotine-based therapies for brain disorders.” Curr Opin Neurobiol 7(5): 674-682.

Lindstrom, J. (1997). “Nicotinic acetylcholine receptors in health and disease.” Molecular Neurobiology 15(2): 193-222.

Lowry, O. H., M. J. Rosebrough, et al. (1951). “Protein measurement with the folin phenol reagent.” Journal biological chemistry 193: 265-275.

Lukas, R. J., J. P. Changeux, et al. (1999). “International Union of Pharmacology. XX. Current status of the nomenclature for nicotinic acetylcholine receptors and their subunits.” Pharmacological Review 51(2): 397-401.

Macklin, K. D., A. D. Maus, et al. (1998). “Human vascular endothelial cells express functional nicotinic acetylcholine receptors.” Journal of Pharmacological and Experimental Therapeutics 287(1): 435-9.

Malpass, J. R., D. A. Hemmings, and A. L. Wallis (1996). Tetrahedron Lett. 37 3911-3914.

Marks, M. J., J. A. Stitzel, et al. (1985). “Time course study of the effects of chronic nicotine infusion on drug response and brain receptors.” Journal of Pharmacological and Experimental Therapeutics 235(3): 619-28.

Maus, A. D., E. F. Pereira, et al. (1998). “Human and rodent bronchial epithelial cells express functional nicotinic acetylcholine receptors.” Molecular Pharmacology 54(5): 779-88.

Paterson, D. and A. Nordberg (2000). “Neuronal nicotinic receptors in the human brain.” Progress in Neurobiolog 61(1): 75-111.

Perry, D. C., M. I. Davila-Garcia, et al. (1999). “Increased nicotinic receptors in brains from smokers: membrane binding and autoradiography studies.” The Journal of Experimental and Clinical Therapeutics 289(3): 1549-1552.

Qian, C, T. Li et al. (1993). “Epibatidine is a nicotinic analgesic.” Eur. J. Pharmacol. 250:R13-R14.

Role, L. W. and D. K. Berg (1996). “Nicotinic receptors in the development and modulation of CNS synapses.” Neuron 16(6): 1077-85.

Sato, K. Z., T. Fujii, et al. (1999). “Diversity of mRNA expression for muscarinic acetylcholine receptor subtypes and neuronal nicotinic acetylcholine receptor subunits in human mononuclear leukocytes and leukemic cell lines.” Neurosci lett 266(1): 17-20.

Schwartz, R. D., K. K. J., et al. (1985). “In vivo regulation of [3H]acetylcholine recognition sites in brain by nicotinic cholinergic drugs.” Journal of Neurochemistry 45(2): 427-33.

Slama, K. (1998). “Tobacco control and prevention, a Guide for Low Income Countries.” IUATLD.

Spande et al., (1992). J. Am. Chem. Soc., 114:3475.

Urien, S. (1995). “Micropharm-K, microcomputer interactive program for the analysis and the simulation of pharmacokinetic processes.” Pharmaceutical Research 12: 1225-1230.

Wonnacott, S. (1997). “Presynaptic nicotinic ACh receptors.” Trends in Neurosciences 20(2): 92-8.

Xu, R., D. Bai et al. (1996a). Bioorg. Med. Chem. Lett. 6:279-282.

Xu, R., G. Chu (1996b). “Epibatidine and Its Analogues” J. Organic Chem. 61:4600-4606.

Zhang, C., L. Gyermek et al. (1997). “Synthesis of Optically Pure Epibatidine Analogs” Tetrahedron Lett. 38 5619-5622.

What is claimed is:

1) A method of measuring the level of a marker indicative of the exposure of a patient to nicotine that comprises
(a) placing epibatidine in contact with leukocytes of said patient;
(b) evaluating the binding of epibatidine to said leukocytes, wherein the amount of bound epibatidine is indicative of the level of said marker on said leukocytes.

2) The method of claim 1, wherein said marker is the epibatidine binding site of a neuronal nicotinic receptor.

3) The method of claim 1, wherein said leukocytes are polymorphonuclear leukocytes.

4) The method of claim 1, wherein said epibatidine is labelled.

5) The method of claim 4, wherein said epibatidine is radiolabelled.

6) The method of claim 5, wherein said epibatidine is [³H]-epibatidine.

7) The method of claim 1, wherein the epibatidine ligand is a synthetic analogue of epibatidine.

8) The method of claim 1, wherein epibatidine binding to leukocytes present in a blood sample establishes whether the subject is a smoker or a non-smoker.

9) The method of claim 1, wherein epibatidine binding to leukocytes present in a blood sample evaluates a subject's tobacco use.

10) The method of claim 1, wherein epibatidine binding to leukocytes present in a blood sample evaluates a subject's tobacco dependence.

11) The method of claim 1, wherein epibatidine binding to leukocytes present in a blood sample evaluates the effects of passive smoke exposure.

12) The method of claim 1, wherein epibatidine binding to leukocytes present in a blood sample monitors diseases or conditions associated with tobacco use.

13) The method of claim 1, wherein epibatidine binding to leukocytes present in a blood sample is the basis for the design of a smoking cessation program.

14) A kit for measuring the level of a marker indicative of the exposure of a patient to nicotine that comprises epibatidine and optionally reagents for evaluating the binding of epibatidine to its binding site.
